(19) United States
(12) Patent Application Publication
(10) Pub. No.: US 2026/0137511 A1
(43) Pub. Date: May 21, 2026

(54) DOCKING STATION WITH SPIRAL STRUTS

(71) Applicant: Edwards Lifesciences Corporation, Irvine, CA (US)

(72) Inventors: Michael G. Valdez, Riverside, CA (US); Amirsepehr Azimian, Costa Mesa, CA (US); Omar Fawzi Azanki, Coto de Caza, CA (US); Anne Bernadette Aragon Alcasid, Costa Mesa, CA (US); David Lyle Leibowitz, Capistrano Beach, CA (US); Tiana Tran, Westminster, CA (US)

(21) Appl. No.: 19/451,237

(22) Filed: Jan. 16, 2026

Related U.S. Application Data
(63) Continuation of application No. PCT/US2024/038242, filed on Jul. 16, 2024.
(60) Provisional application No. 63/514,326, filed on Jul. 18, 2023.

(51) Int. Cl. A61F 2/24

(52) U.S. Cl. CPC A61F 2/2418; A61F 2/2436; A61F 2220/0075; A61F 2230/0006; A61F 2250/0037; A61F 2250/0069; A61F 2250/0098

(57) ABSTRACT

An implant may include an expandable frame configured to expand from a compressed state to an expanded state when deployed, where the expandable frame may include a longitudinal direction along a central axis through a central cavity formed by the expandable frame. The implant may also include multiple struts at a first end of the expandable frame, where the struts extend away from the expandable frame in a series of generally tangential directions away from a circular shape, where the circular shape is disposed within the central cavity, and where the circular shape is normal to the longitudinal direction, and where the circular shape is generally centered around the central axis.

### CROSS REFERENCE TO RELATED APPLICATIONS

This application is a continuation of PCT Patent Application No. PCT/US2024/038242, filed on Jul. 16, 2024, which claims the benefit of U.S. Provisional Patent Application No. 63/514,326, filed Jul. 18, 2023, each of these applications being incorporated herein in its entirety by this specific reference.

### FIELD

The present disclosure relates to implantable adaptor systems for engaging and retaining a prosthetic implant such as a prosthetic heart valve in a lumen of the body, such as a blood vessel or valve of the heart.

### BACKGROUND

Prosthetic heart valves may be used to treat cardiac valvular disorders. The native heart valves (the aortic, pulmonary, tricuspid, and mitral valves) function to prevent backward flow or regurgitation, while allowing forward flow. These heart valves may be rendered less effective by congenital, inflammatory, infectious conditions, etc. Such conditions may eventually lead to serious cardiovascular compromise or death. For many years, doctors attempted to treat such disorders with surgical repair or replacement of the valve during open heart surgery.

A transcatheter technique for introducing and implanting a prosthetic heart valve using a catheter in a manner that is less invasive than open heart surgery may reduce complications associated with open heart surgery. In this technique, a prosthetic valve may be mounted in a crimped state on the end portion of a catheter and advanced through a blood vessel of the patient until the valve reaches the implantation site. The valve at the catheter tip may then be expanded to its functional size at the site of the defective native valve, such as by inflating a balloon on which the valve is mounted or, for example, the valve may have a resilient, self-expanding stent or frame that expands the valve to its functional size when it is advanced from a delivery sheath at the distal end of the catheter. Optionally, the valve may have a balloon-expandable frame, self-expanding frame, a mechanically-expandable frame, and/or a frame expandable in multiple or a combination of ways.

Transcatheter heart valves (THVs) may be appropriately sized for placement inside many native cardiac valves or orifices. However, with larger native valves, blood vessels (e.g., an enlarged aorta), grafts, etc., aortic transcatheter valves might be too small to secure into the larger implantation or deployment site. In this case, the transcatheter valve may not be large enough to sufficiently expand inside the native valve or other implantation or deployment site or the implantation/deployment site may not provide a good seat for the THV to be secured in place. As one example, aortic insufficiency may be associated with difficulty securely implanting a THV in the aorta and/or aortic valve. Accordingly, there exists a need for improved systems and methods of securing a THV in a relatively large diameter blood vessel or annulus.

### SUMMARY

Certain embodiments of the disclosure pertain to docking stations, frame adaptors, prestents, and the like for engaging and retaining a prosthetic implant such as a prosthetic heart valve in a lumen of the body, such as a blood vessel or valve of the heart. In a representative embodiment, an implant may include an expandable frame configured to expand from a compressed state to an expanded state when deployed, where the expandable frame may include a longitudinal direction along a central axis through a central cavity formed by the expandable frame. The implant may also include multiple struts at a first end of the expandable frame, where the struts extend away from the expandable frame in a series of generally tangential directions away from a circular shape, where the circular shape is disposed within the central cavity, and where the circular shape is normal to the longitudinal direction, and where the circular shape is generally centered around the central axis.

The foregoing and other objects, features, and advantages of the described technology will become more apparent from the following detailed description, which proceeds with reference to the accompanying figures.

### BRIEF DESCRIPTION OF THE DRAWINGS

FIG. 1A is a cutaway view of the human heart in a diastolic phase.

FIG. 1B is a cutaway view of the human heart in a systolic phase.

FIG. 2A is a cutaway view of the human heart with an example embodiment of an example docking station positioned in a blood vessel, the inferior vena cava (IVC).

FIG. 2B is an end view of an example docking station and valve showing the valve in an open configuration such that blood may flow through the valve, e.g., when the heart is in a diastolic phase.

FIG. 2C is an end view of the docking station and valve of FIG. 2B showing the valve in a closed configuration, e.g., when the heart is in a systolic phase.

FIG. 3A is a sectional view of an example embodiment of a docking station with an example transcatheter valve disposed inside the docking station.

FIG. 3B is a top view of the docking station and valve illustrated by FIG. 3A.

FIG. 3C is a perspective view of an example embodiment of a docking station that illustrates an example of a frame portion that may be used in the docking station of FIGS. 3A-3B.

FIG. 3D is a sectional view of the docking station illustrated by FIG. 3A where the transcatheter valve shown is representative of a leaflet type transcatheter valve.

FIGS. 4A and 4B schematically illustrate deployment of a docking station.

FIGS. 4C and 4D schematically illustrate deployment of a valve in the docking station.

FIG. 5 illustrates components of another example implant configured to dock and/or support one or more prosthetic valves and/or valve components.

FIG. 6 illustrates components of an additional example implant configured to dock and/or support one or more prosthetic valves and/or valve components.

FIG. 7A illustrates a side view of components of another example implant configured to dock and/or support one or more prosthetic valves and/or valve components.

FIGS. 7B-7F illustrate an example deployment of the implant illustrated in FIG. 7A.

FIG. 8 illustrates a top-down view of components of another example implant configured to dock and/or support one or more prosthetic valves and/or valve components.

FIG. 9A illustrates a portion of the implant of FIG. 8.

FIG. 9B illustrates the implant of FIG. 8 with various measurements and shapes illustrated.

FIG. 10 illustrates an example of the implant of FIG. 8 when cut or etched from a cylindrical tube.

FIG. 11 illustrates another example of an implant configured to dock and/or support one or more prosthetic valves and/or valve components.

FIGS. 12A-12C illustrate examples of struts of an implant.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

It should be understood that the disclosed embodiments may be adapted for delivery and implantation in any of the native annuluses and blood vessels of the heart (e.g., the pulmonary, mitral, and tricuspid annuluses, the inferior and superior vena cava, etc.), and may be used with any of various delivery approaches (e.g., retrograde, antegrade, transseptal, transventricular, transatrial, etc.).

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods may be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, may be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

In the context of the present application, the terms “lower” and “upper” are used interchangeably with the terms “inflow” and “outflow”, respectively. Thus, for example, typically the lower end of a valve or docking station as depicted in the figures is its inflow end and the upper end of the valve or docking station is its outflow end unless explicitly described otherwise.

As used herein, the term “proximal” refers to a position, direction, or portion of a device that is closer to the user (e.g., clinician) and further away from the implantation site and/or body lumen orifice. As used herein, the term “distal” refers to a position, direction, or portion of a device that is further away from the user and closer to the implantation site and/or body lumen orifice. Thus, for example, proximal motion of a device is motion of the device toward the user, while distal motion of the device is motion of the device away from the user.

The terms “longitudinal” and “axial” refer to an axis extending in the upstream and downstream directions, or in the proximal and distal directions, unless otherwise expressly defined.

Although there are alternatives for various components, features, parameters, operating conditions, etc., set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part may become a “lower” part simply by turning the object over. Nevertheless, it is still the same part and the object remains the same. As used herein, “and/or” means “and” or “or”, as well as “and” and “or”.

As used herein, the terms “integrally formed” and “unitary construction” refer to a construction that does not require any sutures, fasteners, or other securing means to attach two portions of the construction together.

### Examples of Disclosed Technology

The present disclosure pertains to valve adapter/docking station/landing zone/prestent technology for implanting a prosthetic heart valve, such as a transcatheter heart valve, in a lumen or valve of the heart where the diameter of the lumen or valve is significantly greater than the functional diameter of the prosthetic valve. In certain examples, the docking station may comprise a radially expandable and collapsible frame formed from a plurality of struts, and including a valve seat within the frame configured to receive an expandable prosthetic valve. In certain embodiments, the valve seat may comprise a plurality of struts coupled to the frame and angled inwardly toward the longitudinal axis of the frame. The valve seat may be configured to engage and retain prosthetic valves of a variety of types and sizes. The outer aspect of the docking station frame may engage the surrounding tissue of the native lumen and form a seal, and the valve seat may engage and retain the prosthetic heart valve within the docking station. In certain embodiments, the frame may comprise a sealing member configured to form a seal between the frame and the surrounding anatomy without substantially interfering with blood flow entering the upstream portions of the frame, such as adjacent the ostia of the hepatic veins when implanted in the inferior vena cava.

In certain embodiments, the struts of the valve seat may form valve seat frame cells of the frame. In certain embodiments, struts and/or cells of the valve seat may comprise free end portions/apices, which may be disposed within the lumen of the docking station frame and define a reduced diameter portion configured to engage and retain a prosthetic heart valve. In certain embodiments, the struts of the valve seat may be coupled to the docking station frame at frame junctions, and the free end portions/apices of the valve seat may be offset from the frame junctions in a downstream direction toward the outflow end of the frame. This reduce or minimize the length of the prosthetic valve that protrudes or extends distally or in the downstream direction from the docking station. In certain embodiments, the struts of the valve seat may be wholly disposed within the docking station frame, or the free end portions/apices of the valve seat may define a downstream-most end of the docking station frame.

In certain embodiments, the docking station frame may comprise a plurality of circumferentially-arranged longitudinal struts. The longitudinal struts may reduce or prevent foreshortening of the frame between the collapsed and expanded configuration. This may facilitate more accurate and/or predictable deployment of the docking station from the collapsed delivery configuration. The longitudinal struts may also facilitate recapture of the docking station frame from a partially deployed state by limiting an angle formed by the flared, partially deployed portion of the frame and the longitudinal axis of the delivery apparatus. The longitudinal struts may also strengthen the frame and reduce or eliminate infolding or invagination of the frame during recapture.

In certain embodiments, the docking station frame may comprise a plurality of free end portions or apices arranged circumferentially around the frame. In certain embodiments, the free apices may be located between pairs of adjacent longitudinal struts. In certain embodiments, the free apices may be proximal and/or distal apices of frame cells defined between pairs of longitudinal frame struts. In certain embodiments, the frame cells may be axially spaced apart from each other. The free apices may be configured to engage the surrounding tissue of a body lumen in which the docking station frame is implanted to prevent frame movement/migration/rotation relative to the body lumen.

In some embodiments, docking stations/devices for prosthetic valves or THVs are illustrated as being used within the superior vena cava (SVC), inferior vena cava (IVC), or both the SVC and the IVC, although the docking stations/devices (e.g., docking station/device 10, other docking stations/devices described herein, modified versions of the docking stations, etc.) may be used in other areas of the anatomy, heart, or vasculature, such as the tricuspid valve, the pulmonary valve, the pulmonary artery, the aortic valve, the aorta, the mitral valve, or other locations. The docking stations/devices described herein may be configured to compensate for the deployed transcatheter valve or THV being smaller and/or having a different geometrical shape than the space (e.g., anatomy/heart/vasculature/etc.) in which it is to be placed. For example, the native anatomy (e.g., the IVC) may be oval, egg shaped, or another shape, while the prosthetic valve or THV may be cylindrical.

Various embodiments of docking stations/devices and examples of prosthetic valves or transcatheter valves are disclosed herein, and any combination of these options may be made unless specifically excluded. For example, any of the docking stations/devices disclosed, may be used with any type of valve, and/or any delivery system, even if a specific combination is not explicitly described. Likewise, the different constructions and features of docking stations/devices and valves may be mixed and matched, such as by combining any docking station type/feature, valve type/feature, covering/sealing element, etc., even if not explicitly disclosed. In short, individual components of the disclosed systems may be combined unless mutually exclusive or physically impossible.

For the sake of uniformity, in the present disclosure the docking stations are typically depicted such that the right atrium end (e.g., the outflow end) is up, while the ventricular end or IVC end (e.g., the inflow end) is down unless otherwise indicated.

### First Representative Embodiment

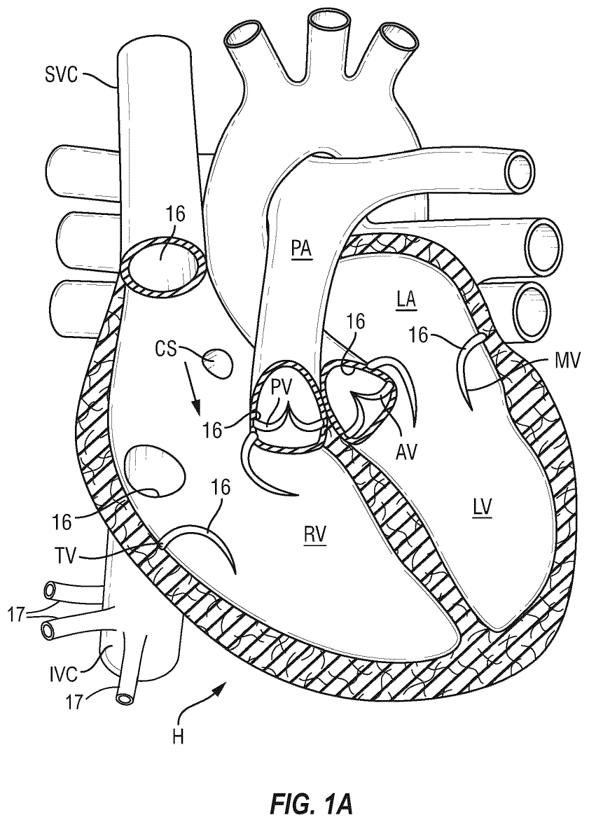

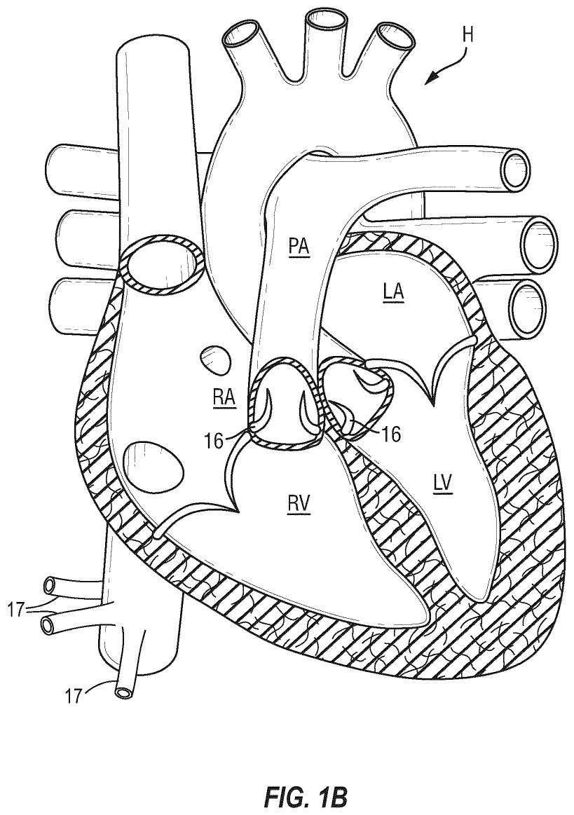

FIGS. 1A and 1B are cutaway views of the human heart (H) in diastolic and systolic phases, respectively. The right ventricle (RV) and left ventricle (LV) are separated from the right atrium (RA) and left atrium (LA), respectively, by the tricuspid valve (TV) and the mitral valve (MV); i.e., the atrioventricular valves. Additionally, the aortic valve (AV) separates the left ventricle (LV) from the ascending aorta (not identified) and the pulmonary valve (PV) separates the right ventricle from the pulmonary artery (PA). Each of these valves has flexible leaflets extending inward across the respective orifices that come together or “coapt” in the flowstream to form one-way, fluid-occluding surfaces. The docking stations and valves of the present application are described, for illustration, primarily with respect to the inferior vena cava (IVC), superior vena cava (SVC), and aorta/aortic valve. A defective aortic valve, for example, may be a stenotic aortic valve and/or suffer from insufficiency and/or regurgitation. The blood vessels, such as the aorta, IVC, SVC, pulmonary artery, may be healthy or may be dilated, distorted, enlarged, have an aneurysm, or be otherwise impaired. Anatomical structures of the right atrium RA, right ventricle RV, left atrium LA, and left ventricle LV will be explained in greater detail. The devices described herein may be used in various areas whether explicitly described herein or not, e.g., in the IVC and/or SVC, in the aorta (e.g., an enlarged aorta) as treatment for a defective aortic valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The hepatic veins 17 carry blood from the liver to the inferior vena cava IVC. The coronary sinus (CS) is a collection of veins joined together to form a large vessel that collects deoxygenated blood from the heart muscle (myocardium), and delivers it to the right atrium RA. During the diastolic phase, or diastole, seen in FIG. 1A, the deoxygenated blood from the IVC, SVC, and CS that has collected in the right atrium RA passes through the tricuspid valve TV and into the RV as the right ventricle RV expands. In the systolic phase, or systole, seen in FIG. 1B, the right ventricle RV contracts to force the deoxygenated blood collected in the RV through the pulmonary valve PV and pulmonary artery into the lungs.

The devices described herein may be used to supplement the function of a defective tricuspid valve and/or to prevent too much pressure from building up in the RA. During systole, the leaflets of a normally functioning tricuspid valve TV close to prevent the venous blood from regurgitating back into the right atrium RA. When the tricuspid valve does not operate normally, blood may backflow or regurgitate into the right atrium RA, the inferior vena cava IVC, the superior vena cava SVC, and/or other vessels in the systolic phase. Blood regurgitating backward into the right atrium increases the volume of blood in the atrium and the blood vessels that direct blood to the heart. This may cause the right atrium to enlarge and cause blood pressure to increase in the right atrium and blood vessels, which may cause damage to and/or swelling of the liver, kidneys, legs, other organs, etc. A transcatheter valve (THV) implanted in the inferior vena cave IVC and/or the superior vena cava SVC may prevent or inhibit blood from backflowing into the inferior vena cave IVC and/or the superior vena cava SVC during the systolic phase.

The length L, diameter D, and curvature or contour may vary greatly between the superior vena cava SVC and inferior vena cava IVC of different patients. The relative orientation and location of the IVC and/or SVC may also vary between patients. Further, the size or diameter D may vary significantly along the length L of an individual IVC and/or SVC. Also, the anatomy of the IVC and/or SVC is soft, flexible, and dynamic as compared to other cardiac vessels, such as the aorta. This softer, more flexible, and/or more dynamic (moving and/or shape changing) characteristic of the IVC and SVC make it more difficult for a transcatheter valve frame or a docking station that supports a transcatheter valve to anchor in the IVC and/or the SVC than in the aorta. Further, other regions or other vasculature in other areas of the body and across patients where docking stations could be used may also vary significantly in shape and size.

The left atrium LA receives oxygenated blood from the left and right pulmonary veins, which then travels through the mitral valve to the left ventricle. During the diastolic phase, or diastole, seen in FIG. 1A, the oxygen rich blood that collects in the left atrium LA passes through the mitral valve MV and into the left ventricle LV as the left ventricle LV expands. In the systolic phase, or systole, seen in FIG. 1B, the left ventricle LV contracts to force the oxygen rich blood through the aortic valve AV and aorta into the body through the circulatory system. In certain embodiments, the devices described herein may be used to supplement or replace the function of a defective aortic valve. For example, the devices herein may be particularly effective for treating aortic insufficiency. During diastole, the leaflets of a normally functioning aortic valve AV close to prevent the oxygen rich blood from regurgitating back into the left ventricle LV. When the aortic valve does not operate normally, blood backflows or regurgitates into the left ventricle LV. A THV implanted in the aortic valve helps prevent or inhibit blood from backflowing into the left ventricle LV during the diastole phase. The length L, diameter, D, and curvature or contour of the aortic root may vary greatly between different patients, especially if the aorta is dilated, distorted, or enlarged. Further, the size or diameter D may vary significantly along the length L of an individual aorta.

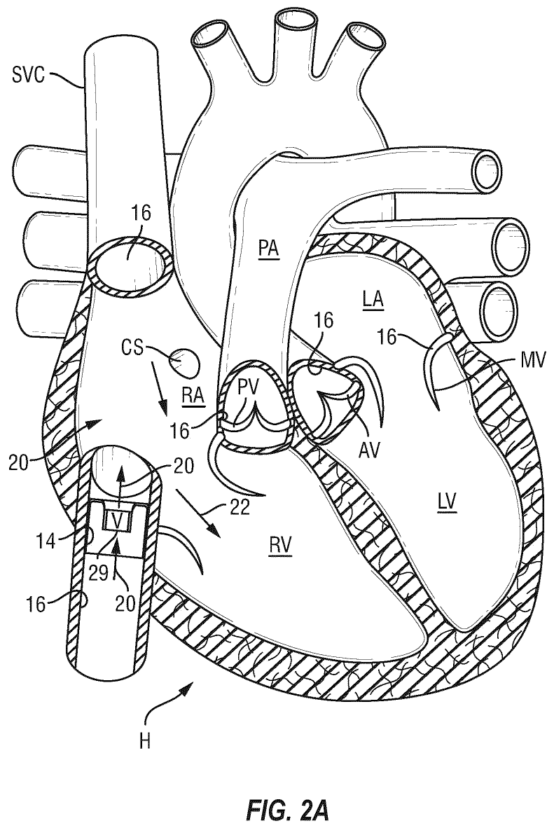

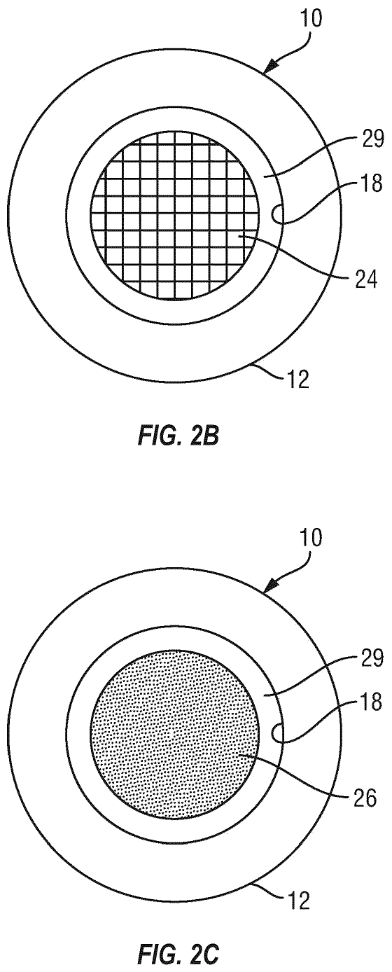

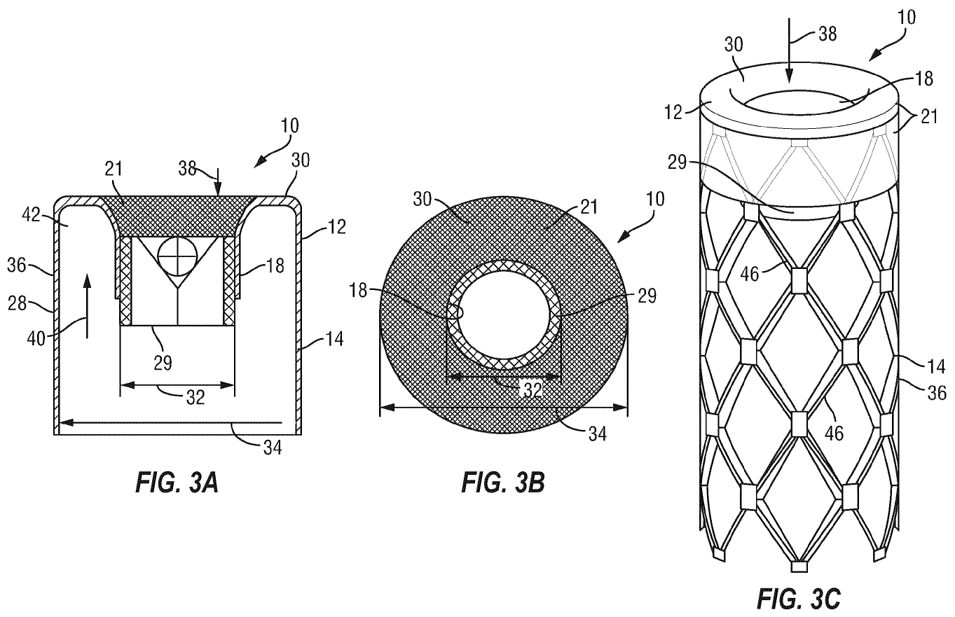

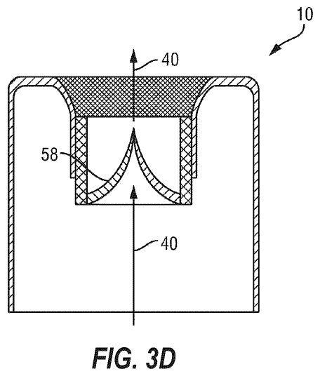

Referring to FIGS. 2A, 3A, 3B, and 3C, in some embodiments an expandable docking station/device/valve adapter/landing zone/prestent 10 includes one or more sealing portions 12, a valve seat 18, and one or more retaining portions 14. The sealing portion(s) 12 provide a seal between the docking station 10 and an interior surface 16 (see FIG. 2A) of the circulatory system. The valve seat 18 provides a supporting surface for implanting or deploying a valve 29 in the docking station 10 after the docking station 10 is implanted in the circulatory system. Optionally, the docking station 10 and the valve 29 may be integrally formed, for example, in one embodiment, the valve seat 18 may be omitted. When integrally formed, the docking station 10 and the valve 29 may be deployed as a single device, rather than first deploying the docking station 10 and then deploying the valve 29 into the docking station. Any of the docking stations and/or valve seats 18 described herein may be provided or formed with an integrated valve 29.

The retaining portion 14 helps retain the docking station 10 and the valve 29 at the implantation position or deployment site in the circulatory system. The retaining portion 14 may take a wide variety of different forms. In some embodiments, the retaining portion 14 includes friction enhancing features that reduce or eliminate migration of the docking station 10. The friction enhancing features may take a wide variety of different forms. For example, the friction enhancing features may comprise barbs, spikes, texturing, adhesive, and/or a cloth or polymer cover with high friction properties on the retaining portions 14. Such friction enhancing features may also be used on any of the various docking stations or retaining portions described herein.

Expandable docking station 10 and valve 29 as described in the various embodiments herein are also representative of a variety of docking stations and/or valves described herein or that might be known or developed, e.g., a variety of different types of valves could be substituted for and/or used as valve 29 in the various docking stations.

FIGS. 2A, 2B, and 2C illustrate a representative example of the operation of the docking stations 10 and valves 29 disclosed herein. In the example of FIGS. 2A, 2B, and 2C, the docking station 10 and valve 29 are deployed in the inferior vena cava IVC. However, the docking station 10 and valve 29 may be deployed in any interior surface within the heart or a lumen of the body. For example, the various docking stations and valves described herein may be deployed in the superior vena cava SVC, the tricuspid valve TV, the pulmonary valve PV, pulmonary artery, the mitral valve MV, the aortic valve AV, aorta, or other vasculature/lumens in the body.

FIGS. 2A and 2B illustrate the valve 29, docking station 10 and heart H, when implanted in the IVC and the heart H is in the diastolic phase. When the heart is in the diastolic phase, the valve 29 opens. Blood flows from the inferior vena cava IVC and the superior vena cava SVC, into the right atrium RA. The blood that flows from the inferior vena cava IVC flows through the docking station 10 and valve 29 as indicated by arrows 20. Also, while in the diastolic phase, blood in the right atrium flows through the tricuspid valve TV, and into the right ventricle RV and valve as indicated by arrows 22. FIG. 2B illustrates space 24 that represents the valve 29 being open when the heart is in the diastolic phase. A variety of types of valves may be used that may open and close in a variety of ways (e.g., including valves with leaflets of tissue that open then coapt to close), so the drawings are meant to be representative of a variety of valves that may operate in different ways. FIG. 2B does not show the interface between the docking station 10 and the inferior vena cava to simplify the drawing. The cross-hatching in FIG. 2B represents blood flow through the valve 29. In some embodiments, blood is prevented or inhibited from flowing between the inferior vena cava IVC and the docking station 10 by the sealing portion 12 and blood is prevented or inhibited from flowing between the docking station 10 and the valve by implanting or seating the valve in the seat 18 of the docking station 10. In this example, blood only substantially flows or is only able to flow through the valve 29 when the valve is open (e.g., in certain embodiments, only when the heart is in the diastolic phase).

FIG. 2C illustrates the valve 29 and docking station 10, when the valve 29 is closed (e.g., when implanted in the IVC and the heart H is in the systolic phase). When implanted in the IVC and the heart is in the systolic phase, the valve 29 closes. Blood is prevented from flowing from the right atrium RA into the inferior vena cava IVC by the valve 29 being closed. As such, the closed valve 29 prevents any blood that regurgitates through the tricuspid valve TV during the systolic phase from being forced into the inferior vena cava IVC. The solid area 26 in FIG. 2C represents the valve 29 being closed (e.g., in certain embodiments, when the heart is in the systolic phase). FIG. 2C is meant to be representative of a variety of valves, even though those valves may close in different ways.

In some embodiments, the docking station 10 acts as an isolator that prevents or substantially prevents radial outward forces of the valve 29 from being transferred to the inner surface 16 of the circulatory system. In one embodiment, the docking station 10 includes a valve seat 18 that resists expansion, e.g., is not expanded radially outwardly (e.g., the diameter of the valve seat does not increase) or is not substantially expanded radially outward (e.g., the diameter of the valve seat increases by less than 4 mm) by the radially outward force of the transcatheter valve or valve 29. The valve seat may be configured such that expansion of a THV/valve 29 increases the diameter of the valve seat only to a diameter less than an outer diameter of the docking station 10 when the docking station is implanted. Retaining portions 14 and sealing portions 12 may be configured to impart only relatively small radially outward forces on the inner surface 16 of the circulatory system (as compared to the radially outward force applied to the valve seat 18 by the valve 29). Having a valve seat 18 that is stiffer or less radially expansive than the outer portions of the docking station (e.g., retaining portions 14 and sealing portions 12), as in the various docking stations described herein, provides many benefits, including allowing a THV/valve 29 to be implanted in vasculature or tissue of varying strengths, sizes, and/or shapes. The outer portions of the docking station may better conform to the anatomy (e.g., vasculature, tissue, heart, etc.) without putting too much pressure on the anatomy, while the THV/valve 29 may be firmly and securely implanted in the valve seat 18 with forces that will prevent or mitigate the risk of migration or slipping.

The docking station 10 may include any combination of one or more than one different types of valve seats 18, retaining portions 14, and/or sealing portions 12. For example, the valve seat 18 may be a separate component that is attached to the frame 28 of the docking station 10, while the sealing portion is integrally formed with the frame 28 of the docking station. Also, the valve seat 18 may be a separate component that is attached to the frame 28 of the docking station 10, while the sealing portion 12 is a separate component that is also attached to the frame 28 of the docking station. Optionally, the valve seat 18 may be integrally formed with the frame 28 of the docking station 10, while the sealing portion is integrally formed with the frame 28 of the docking station. Further, the valve seat 18 may be integrally formed with the frame 28 of the docking station 10, while the sealing portion is a separate component that is attached to the frame 28 of the docking station 10.

The sealing portion 12, the valve seat 18, and one or more retaining portions 14 of the various docking stations herein may take a variety of different forms and characteristics. In FIGS. 3A-3C, an expandable frame 28 provides the shape of the sealing portion 12, the valve seat 18, and the retaining portion 14. The expandable frame 28 may take a wide variety of different forms. The illustrated expandable frame 28 in FIGS. 3A-3C has an end 30 having an inside diameter 32 and an outside diameter 34. An annular or cylindrical outer portion or wall 36 extends downward from the outside diameter 34 of the end 30. An annular or cylindrical valve seat 18 or wall extends downward from the inside diameter 32 of the end 30. In the illustrated example, the expandable frame 28 is an expandable lattice. The expandable lattice may be made in a variety of ways, e.g., with individual wires connected to form the lattice, braiding, cut from a sheet and then rolled or otherwise formed into the shape of the expandable frame, molded, cut from a cylindrical tube (e.g., cut from a nitinol tube), other ways, or a combination of these.

The frame 28 may be made from a highly flexible metal, metal alloy, or polymer. Examples of metals and metal alloys that may be used include, but are not limited to, nitinol and other shape memory alloys, elgiloy, and stainless steel, but other metals and highly resilient or compliant non-metal materials may be used to make the frame 28. These materials may allow the frame to be compressed to a small size, and then when the compression force is released, the frame will self-expand back to its pre-compressed diameter and/or the frame may be expanded by inflation of a device positioned inside the frame. The frame 28 may also be made of other materials and be expandable and collapsible in different ways, e.g., mechanically-expandable, balloon-expandable, self-expandable, or a combination of these.

The sealing portions may take a wide variety of different forms. In the example of FIGS. 3A-3C, a covering/material 21 is attached to a portion of the frame 28 to form the sealing portion 12. However, the sealing portion 12 may be formed in a wide variety of other ways. The covering/material 21 may be a fabric material, polymer material, or other material. The sealing portion 12 may take any form that prevents or inhibits the flow of blood from flowing around the outside surface of the valve 29 and through the docking station. In the example of FIGS. 3A, 3B, and 3C, the sealing portion 12 comprises a covering/material 21 (e.g., a fabric or other covering material that may be the same as or similar to other coverings/materials described herein) that extends up to the valve seat 18. The covering/material 21 may be shaped and positioned in a variety of ways, e.g., the covering/material may be configured to partially cover the valve seat 18, entirely cover the valve seat 18, or not cover the valve seat 18 when the frame 28 is expanded. The covering/material 21 (e.g., fabric or other covering material) that forms the sealing portion 12 may also extend radially outward, covering the end 30 of the frame 28, and may optionally extend (e.g., longitudinally, downward, etc.) to cover at least a portion of the annular outer portion or wall 36. The sealing portion 12 provides a seal between the docking station 10 and an interior surface 16 (see FIG. 2) of the circulatory system. That is, the sealing portion 12 and the closed valve 29 prevent or inhibit blood from flowing in the direction indicated by arrow 38. In the example of FIGS. 3A and 3B, blood is not inhibited from flowing in the direction indicated by arrow 40 into the area 42 between the valve seat 18 and the annular outer portion or wall 36.

The valve seat may take a wide variety of different forms. The valve seat 18 is a portion of the frame 28 in the example of FIGS. 3A-3C. However, the valve seat 18 may be formed separately from the frame 28. The valve seat 18 may take any form that provides a supporting surface for implanting or deploying a valve 29 in the docking station 10 after the docking station 10 is implanted in the circulatory system. The valve seat may optionally be reinforced with a reinforcing material (e.g., a suture, wire, band, collar, etc. that may circumscribe the valve seat or a portion of the valve seat). The valve 29 is schematically illustrated in FIG. 3A to indicate that the valve 29 may take a wide variety of different forms. FIG. 3D illustrates the more specific example where the valve 29 is a leaflet type THV, such as the SAPIEN® 3 valve available from Edwards Lifesciences Corporation, including a plurality of leaflets 58. In some embodiments, a valve 29 is integrated with or replaces the valve seat 18, such that docking station 10 is configured as a transcatheter valve that is delivered as a single unit in the same step (as opposed to first implanting a docking stations and subsequently implanting a separate valve/THV in the docking station). Optionally, any of the docking stations described herein may be formed as a valve or THV, e.g., with valve tissue or other valve material integrated into the docking station.

The retaining portions 14 may take a wide variety of different forms. For example, the retaining portion(s) 14 may be any structure that sets the position of the docking station 10 in the circulatory system. For example, the retaining portion(s) 14 may press against or into the inside surface 16 or contour/extend around anatomical structures of the circulatory system to set and maintain the position of the docking station 10. The retaining portion(s) 14 may be part of or define a portion of the body and/or sealing portion of the docking station 10 or the retaining portion(s) 14 may be a separate component that is attached to the body of the docking station. The docking station 10 may include a single retaining portion 14 or two, or more than two retaining portions. The retaining portion(s) 14 may include friction enhancing features as discussed above.

In the example of FIGS. 3A-3C, the retaining portion 14 comprises the annular outer portion or wall 36 of the frame 28. A shape set (e.g., a programmed shape of a shape memory material) of annular outer portion or wall 36 may bias the annular outer portion or wall 36 radially outward and into contact with/against the interior surface 16 of the circulatory system to retain the docking station 10 and the valve 29 at the implantation position. In the illustrated embodiment, the retaining portion 14 is elongated to allow a relatively small force to be applied to a large area of the interior surface 16, while the valve 29 may apply a relatively large force to the valve seat 18. For example, the length of the retaining portion 14 may be twice, three times, four times, five times, or greater than five times the outside diameter of the transcatheter valve. Applying a small radially outward force over a larger area may be sufficient to securely hold the docking station in place, and this design/configuration may allow the docking station to conform to the unique shape/size of the anatomy and avoid/reduce the likelihood of damaging relatively weaker native tissue. Thereby the valve 29 may be securely held in a variety of locations and anatomies (e.g., the docking station of FIGS. 3A-D is usable in the IVC, SVC, aorta, etc.).

In certain examples, the retaining portion 14 may comprise the annular outer portion or wall 36 of the frame 28. A shape set (e.g., a programmed shape of a shape memory material) of annular outer portion or wall 36 may bias the annular outer portion or wall 36 radially outward and into contact with/against the interior surface 16 of the aorta to retain the docking station 10 and the valve 29 at the implantation position. In certain examples, the shape set may also be selected to substantially match the shape of a portion of the aorta. The retaining portion 14 may be elongated to allow a relatively small force to be applied to a large area of the interior surface 16, while the valve 29 may apply a relatively large force to the valve seat 18, as discussed above.

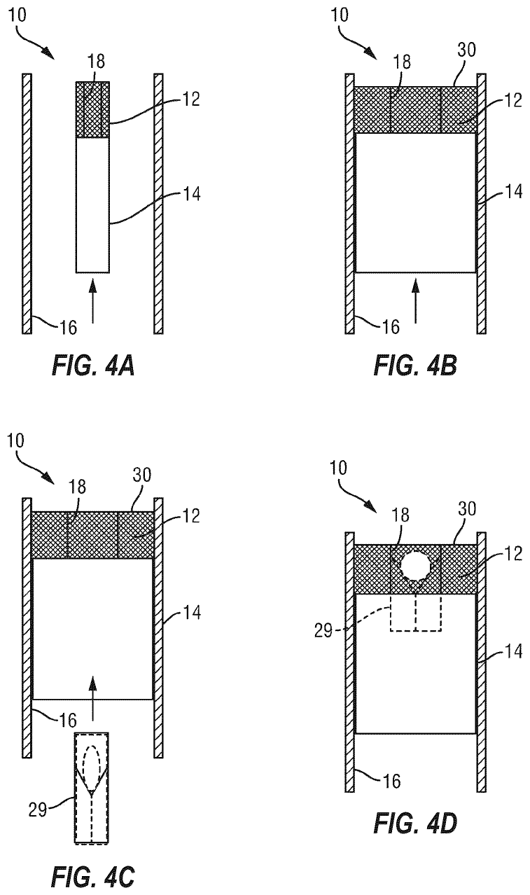

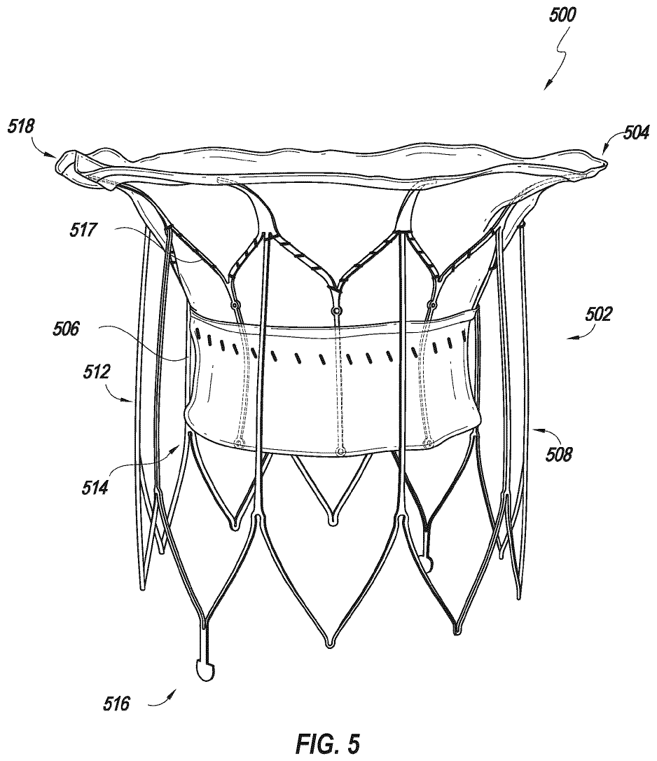

FIGS. 4A-4D schematically illustrate an example deployment of the docking station 10 and valve 29 in the circulatory system. Referring to FIG. 4A, the docking station 10 is in a compressed form/configuration and is introduced to a deployment site in the circulatory system. For example, the docking station 10, may be positioned at a deployment site in a SVC, IVC, aorta, or other location. Referring to FIG. 4B, the docking station 10 is expanded in the circulatory system such that the sealing portion(s) 12 and the retaining portion(s) 14 engage the inside surface 16 of a portion of the circulatory system. The docking station may be self-expanding, and may be advanced from a delivery capsule into the expanded state, or plastically expandable such that it may be expanded using a balloon or other expansion device. Referring to FIG. 4C, after the docking station 10 is deployed, the valve 29 is in a compressed form and is introduced into the valve seat 18 of the docking station 10. Referring to FIG. 4D, the valve 29 is expanded in the docking station, such that the valve 29 engages the valve seat 18 and the seat 18 of the docking station supports the valve. The docking station 10 allows the valve 29 to operate within the expansion diameter range for which it is designed. In the examples depicted herein, the docking station 10 is longer than the valve. However, in some embodiments the docking station 10 may be the same length or shorter than the length of the valve 29. Similarly, the valve seat 18 may be longer, shorter, or the same length as the length of the valve 29. Any of the docking station embodiments described herein may be deployed in the manner described above.

### Second Representative Embodiment

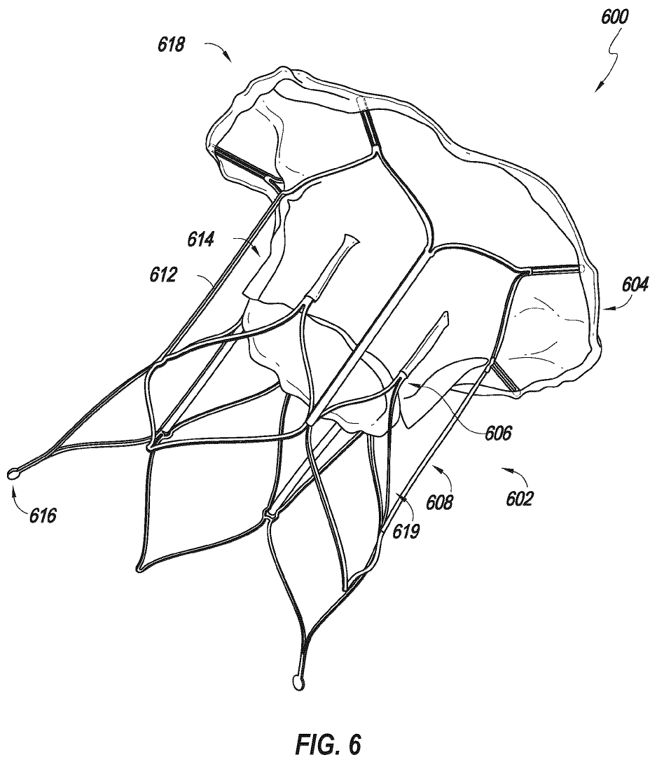

FIG. 5 illustrates components of another example implant 500 configured to dock and/or support one or more prosthetic valves and/or valve components in accordance with one or more instances. The implant 500 may comprise a frame 502 and/or a sealing element 504, which may include a skirt, covering, and/or similar device. The frame 502 may be configured to form an inner frame 506 and/or an outer frame 508. The inner frame 506 may form a first diameter that is less than a second diameter of the outer frame 508. The inner frame 506 and outer frame 508 may be extensions of a common device and/or may extend from each other.

The frame 502 may comprise a wire frame formed by a network of struts 512 (e.g., wires, cords, and/or bars) forming one or more cells 514. The frame 502 may mostly comprise empty space and/or cells 514 between the struts 512. For example, the struts 512 may be generally thin and/or may be spaced apart to create relatively large gaps between the struts 512, as shown in FIG. 5. In some examples, the outer frame 508 may comprise a series of longitudinally extending struts 512 aligned in series and/or in parallel around a circumference of the implant 500. Each of the struts 512 may join to one or more adjacent struts 512 at a proximal end 516 of the implant 500 and/or may join with one or more adjacent struts 512 and/or with the inner frame 506 at a distal end 518 of the implant 500.

The inner frame 506 may similarly comprise a network of generally thin, elongate, and/or spaced apart struts 512. In some examples, one or more struts 512 of the inner frame 506 may extend generally in series and/or in parallel longitudinally along a length of the implant 500. The one or more struts 512 of the inner frame 506 may be disposed between struts 512 of the outer frame 508.

In some examples, the outer frame 508 may have a generally cylindrical form and/or may at least partially enclose a complete circumference of the inner frame 506 and/or of a portion of the sealing element 504. The outer frame 508 may comprise a network of struts 512, which may include wires, arms, bars, cords, walls, and/or similar components forming one or more cells 514 and/or openings through the outer frame 508. The one or more cells 514 may be configured to allow blood flow through the outer frame 508. The cells 514 may have any suitable shape and/or size. The outer frame 508 may form generally elongate cells 514 extending approximately an entire length of the outer frame 508 and/or from a proximal end 516 to a distal end 518 of the implant 500. The one or more cells 514 may have triangular forms at end points of the one or more cells 514. However, the one or more struts 512 may form cells 514 having different shapes. For example, the struts 512 may be configured to form generally rectangular and/or diamond-shaped cells 514. While the struts 512 are shown having generally thick structures, the one or more struts 512 may have wire-like and/or generally thin forms. In some examples, the inner frame 506 and/or outer frame 508 may be configured to maintain a uniform structure and/or strut 512 pattern along a length of the frame 502.

The implant 500 may be configured for delivery and/or placement at an SVC and/or IVC of a heart. For example, the implant 500 may be configured for placement at or near an in-flow junction of the SVC and/or IVC to the right atrium of the heart.

At a distal end 518 of the implant 500, the outer frame 508 and inner frame 506 may join together and/or the inner frame 506 may extend away from the outer frame 508 and/or along an inner lumen formed by the outer frame 508. In some examples, the outer frame 508 and inner frame 506 may both have a generally flared and/or conical form at or near the distal end 518 of the implant 500. The inner frame 506 may have a flaring angle that is greater than the outer frame 508 such that the diameter of the inner frame 506 may be less than the diameter of the outer frame 508. The inner frame 506 may be configured to extend along at least a portion of the length of the outer frame 508.

The flared end (e.g., distal end 518) of the outer frame 508 and/or inner frame 506 may be configured to engage an atrium and/or other chamber when implanted. The sealing element 504 may be configured to extend along the inner frame 506 and/or may be configured to wrap around the outer frame 508 and/or extend between the outer frame 508 and the native tissue. The outer frame 508 may be configured to extend into the IVC and/or other blood vessel and/or the sealing element 504 may be configured to extend from the outer frame 508 to an inner surface of the inner frame 506. The sealing element 504 may be configured for engagement with a prosthetic valve and/or other implant. For example, the sealing element 504 may be configured to provide a mounting surface for a prosthetic valve and/or may be configured to securely hold the prosthetic valve. The sealing element 504 may be configured to extend along only a portion of the implant 500 such that the sealing element 504 may not extend across one or more branching vessels of the blood vessel.

In some examples, the implant 500 may comprise one or more outward bulbs configured to extend outwardly from the diameter of the outer frame 508 to facilitate anchoring of the implant 500 within a blood vessel.

The frame 502 may comprise one or more downward-extending arms 517 (e.g., extending towards the proximal end 516) configured to form the inner frame 506. For example, the arms 517 may extend downwardly from the outer frame 508 at or near the distal end 518 of the frame 502. The one or more arms 517 may be configured to extend at an acute angle away from the outer frame into the lumen of the frame 502 and/or may extend generally in parallel with the outer frame 508 at or near distal ends of the one or more arms 517.

FIG. 6 illustrates components of another example implant 600 configured to dock and/or support one or more prosthetic valves and/or valve components in accordance with one or more instances. The implant 600 may comprise a frame 602 and/or a sealing element 604, which may include a skirt, covering, and/or similar device. The frame 602 may be configured to form an inner frame 606 and/or an outer frame 608. The inner frame 606 may form a first diameter that is less than a second diameter of the outer frame 608. The inner frame 606 and outer frame 608 may be extensions of a common device and/or may extend from each other.

The frame 602 may comprise a wire frame formed by a network of struts 612 (e.g., wires, cords, and/or bars) forming one or more cells 614. Such struts 612 may be referred to as vertical struts. The frame 602 may mostly comprise empty space and/or cells 614 between the struts 612. For example, the struts 612 may be generally thin and/or may be spaced apart to create relatively large gaps between the struts 612, as shown in FIG. 6. In some examples, the outer frame 608 may comprise a series of longitudinally extending struts 612 aligned in series and/or in parallel around a circumference of the implant 600. Each of the struts 612 may join to one or more adjacent struts 612 at a proximal end 616 of the implant 600 and/or may join with one or more adjacent struts 612 and/or with the inner frame 606 at a distal end 618 of the implant 600.

The inner frame 606 may similarly comprise a network of generally thin, elongate, and/or spaced apart struts 612. In some examples, one or more struts 612 of the inner frame 606 may extend generally in series and/or in parallel longitudinally along a length of the implant 600. The one or more struts 612 of the inner frame 606 may be disposed between struts 612 of the outer frame 608.

In some examples, the outer frame 608 may have a generally cylindrical form and/or may at least partially enclose a complete circumference of the inner frame 606 and/or of a portion of the sealing element 604. The outer frame 608 may comprise a network of struts 612, which may include wires, arms, bars, cords, walls, and/or similar components forming one or more cells 614 and/or openings through the outer frame 608. The one or more cells 614 may be configured to allow blood flow through the outer frame 608. The cells 614 may have any suitable shape and/or size. The outer frame 608 may form generally elongate cells 614 extending approximately an entire length of the outer frame 608 and/or from a proximal end 616 to a distal end 618 of the implant 600. The one or more cells 614 may have triangular forms at end points of the one or more cells 614. However, the one or more struts 612 may form cells 614 having different shapes. For example, the struts 612 may be configured to form generally rectangular and/or diamond-shaped cells 614. While the struts 612 are shown having generally thick structures, the one or more struts 612 may have wire-like and/or generally thin forms. In some examples, the inner frame 606 and/or outer frame 608 may be configured to maintain a uniform structure and/or strut 612 pattern along a length of the frame 602.

The implant 600 may be configured for delivery and/or placement at an SVC and/or IVC of a heart. For example, the implant 600 may be configured for placement at or near an in-flow junction of the SVC and/or IVC to the right atrium of the heart.

At a distal end 618 of the implant 600, the outer frame 608 and inner frame 606 may join together and/or the inner frame 606 may extend away from the outer frame 608 and/or along an inner lumen formed by the outer frame 608. In some examples, the outer frame 608 and inner frame 606 may both have a generally flared and/or conical form at or near the distal end 618 of the implant 600. The inner frame 606 may have a flaring angle that is greater than the outer frame 608 such that the diameter of the inner frame 606 may be less than the diameter of the outer frame 608. The inner frame 606 may be configured to extend along at least a portion of the length of the outer frame 608.

The flared end (e.g., distal end 618) of the outer frame 608 and/or inner frame 606 may be configured to engage an atrium and/or other chamber when implanted. The sealing element 604 may be configured to extend along the inner frame 606 and/or may be configured to wrap around the outer frame 608 and/or extend between the outer frame 608 and the native tissue. The outer frame 608 may be configured to extend into the IVC and/or other blood vessel and/or the sealing element 604 may be configured to extend from the outer frame 608 to an inner surface of the inner frame 606. The sealing element 604 may be configured for engagement with a prosthetic valve and/or other implant. For example, the sealing element 604 may be configured to provide a mounting surface for a prosthetic valve and/or may be configured to securely hold the prosthetic valve. The sealing element 604 may be configured to extend along only a portion of the implant 600 such that the sealing element 604 may not extend across one or more branching vessels of the blood vessel.

In some examples, the implant 600 may comprise one or more outward bulbs configured to extend outwardly from the diameter of the outer frame 608 to facilitate anchoring of the implant 600 within a blood vessel.

The frame 602 may comprise one or more upward-extending arms 619 (e.g., extending towards the distal end 618) configured to form the inner frame 606. For example, the arms 619 may extend downwardly from the outer frame 608 at or near the distal end 618 of the frame 602. The one or more arms 619 may be configured to extend at an acute angle away from the outer frame into the lumen of the frame 602 and/or may extend generally in parallel with the outer frame 608 at or near distal ends of the one or more arms 619.

The arms 619 of the implant 600 may extend upwardly from the proximal end 616 to the distal end 618. For example, the one or more arms 619 may extend at an approximately 45-degree angle from the outer frame 608 at or near the distal end 618 of the implant 600 and/or may extend upwardly generally in parallel with the outer frame 608 along a midsection of the implant 600.

### Third Representative Embodiment

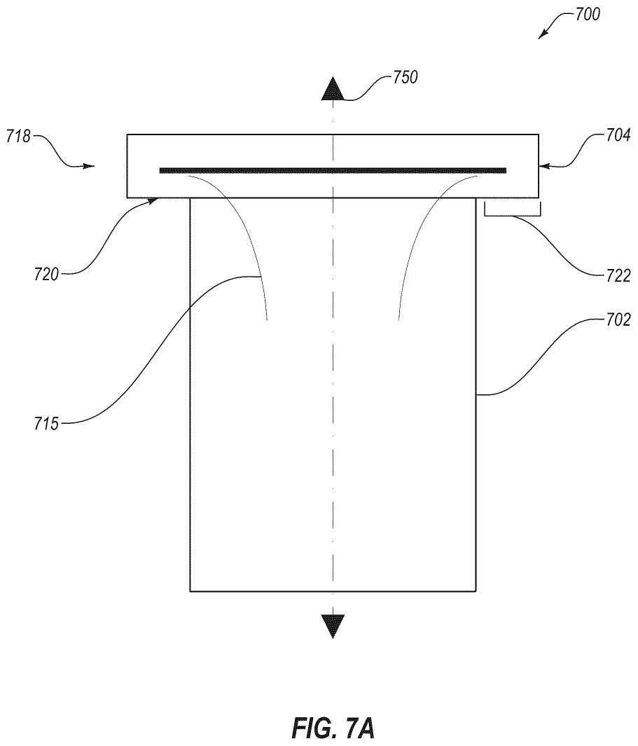

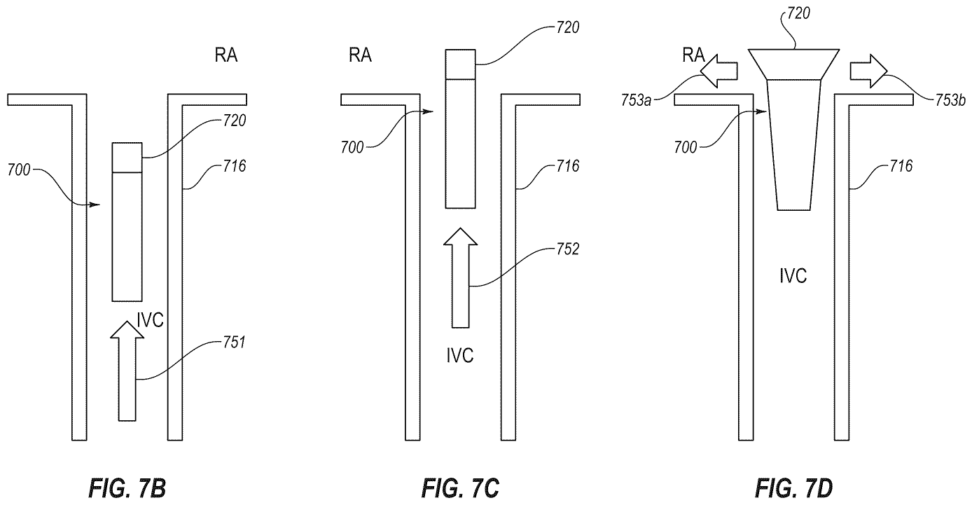

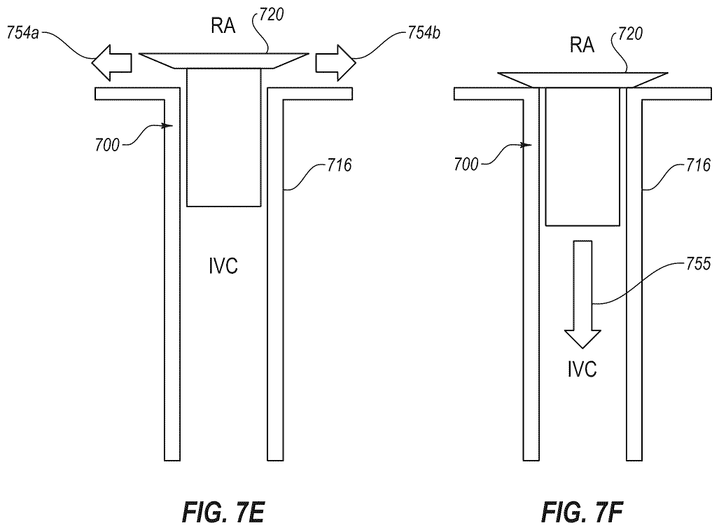

FIG. 7A illustrates a side view of components of another example implant 700 configured to dock and/or support one or more prosthetic valves and/or valve components in accordance with one or more embodiments of the present disclosure. The implant 700 may include a frame 702 and/or a sealing element 704, which may include a skirt, covering, and/or similar device. The frame 702 may be configured to form a valve seat 715. The valve seat 715 may be disposed within a central cavity created by the frame 702. At a distal end 718 of the implant 700, the implant may include a flange 720 to facilitate the sealing element 704 sealing against the anatomy of a patient. For convenience, a central axis 750 of the implant 700 is also illustrated in FIG. 7A.

The flange 720 may provide a projection that extends beyond the remainder of the frame 702. For example, as illustrated in FIG. 7, the flange 720 may produce a lip 722 that is shaped or configured to abut anatomy of the patient. For example, rather than being disposed wholly within the IVC (such as the embodiment illustrated in FIG. 2A), the distal end 718 of the implant 700 may extend into the right atrium with the flange 720 resting against the sidewall of the right atrium proximate where the IVC enters the right atrium (RA). By providing the lip 722, a better seal may be created by the sealing element 704 as compared to embodiments that are radially pressed against the IVC, such as that illustrated in FIG. 2A.

In some embodiments, the shape or profile of the frame 702 or other components forming the flange 720 or flared end at the distal end 718 of the implant 700 may include a similar or comparable profile to that illustrated in FIGS. 5 and/or 6. For example, the frame 702 may include one or more cells with arms extending beyond the remainder of the frame 702 at the distal end 718 of the implant 700.

In some embodiments, the flange 720 may be generally normal to the central axis 750. Additionally or alternatively, the flange 720 may be generally centered around the line depicting the central axis 750.

FIGS. 7B-7F illustrate an example deployment of the implant 700 illustrated in FIG. 7A. The implant 700 may traverse a circulatory system 716 of a patient to arrive at a target deployment location.

As illustrated in FIG. 7B, a catheter (not illustrated) carrying the implant 700 may traverse the circulatory system 716 towards a deployment location. For example, as illustrated in FIG. 7B, it may be desired to deploy the implant 700 between the IVC and the RA. The implant 700 may traverse the circulatory system 716 in a distal direction conveyed by the arrow 751. In some embodiments, the implant 700 may be included in a delivery capsule or may be deployable via a balloon or other deployment mechanism.

As illustrated in FIG. 7C, the implant 700 may begin to pass through the IVC and into the RA as it continues along the distal direction of travel conveyed by the arrow 752.

As illustrated in FIG. 7D, upon reaching the deployment or target location, the implant 700 may be either partially or fully deployed. For example, a sheath or outer covering of the delivery capsule associated with the catheter may be moved proximally relative to the implant 700, exposing the flange 720 and/or other portions of the implant 700. The radial expansion of the implant 700 during deployment is conveyed by the arrows 753a and 753b. While illustrated as being partially within the RA and partially within the IVC, it will be appreciated that the implant 700 may be fully within the RA when it is partially or fully deployed.

As illustrated in FIG. 7E, the implant 700 may be deployed until the flange 720 is substantially or fully deployed. The continued deployment is illustrated by the arrows 754a and 754b. In some embodiments, the implant 700 may be deployed until a radial edge of the flange 720 extends beyond the circumference of the IVC. Such deployment may include full or partial deployment of the flange 720 and/or full or partial deployment of the implant 700.

In some embodiments, the stage of deployment illustrated in FIG. 7E may include the implant 700 being at least 30% deployed, at least 40% deployed, at least 50% deployed, at least 60% deployed, at least 70% deployed, at least 80% deployed, at least 90% deployed, or 100% deployed (or fully deployed). Additionally or alternatively, the stage of deployment illustrated in FIG. 7E may include the flange 720 portion of the implant 700 being at least 30% deployed, at least 40% deployed, at least 50% deployed, at least 60% deployed, at least 70% deployed, at least 80% deployed, at least 90% deployed, or 100% deployed (or fully deployed).

As illustrated in FIG. 7F, after the stage of deployment illustrated in FIG. 7E is reached, the catheter may be drawn back in the proximal direction as illustrated by the arrow 755. Doing so may facilitate the flange 720 seating against the wall of the RA to create a seal.

In some embodiments, after seating the flange 720 against the wall of the RA, the implant 700 may be deployed the remainder of the way to be fully deployed. Additionally, the implant 700 may be disengaged from the catheter and the catheter may be retrieved from the body of the patient. For example, a coupling foot of the frame 702 may be decoupled from an attachment feature of the delivery capsule. Additionally or alternatively, after the implant 700 is fully deployed, the valve may traverse the circulatory system 716 of the patient and be affixed to the implant 700 (such as by being coupled to a catheter in a valve delivery capsule, traversing the circulatory system 716 of the patient until the valve may be deployed from the valve delivery capsule and affixed to the implant 700, and the catheter and valve delivery capsule retrieved).

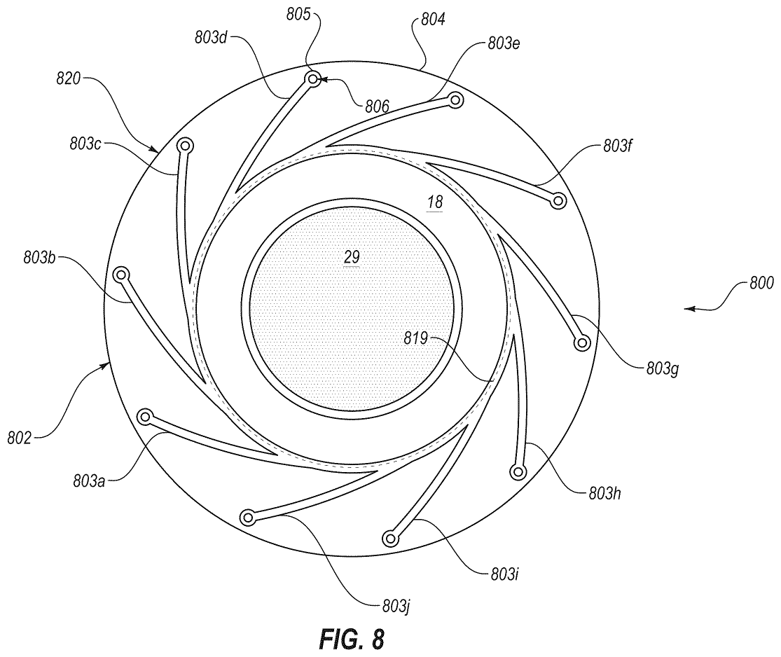

FIG. 8 illustrates a top-down view of components of another example implant 800 configured to dock and/or support one or more prosthetic valves 29 and/or valve components in accordance with one or more embodiments of the present disclosure. The implant 800 may include a frame 802 and/or a sealing element 804, which may include a skirt, covering, and/or similar device. The frame 802 may be configured to form a valve seat 18 and a flange 820 similar or comparable to the flange 720 illustrated in FIG. 7.

The implant 800 may be similar or comparable in some regards to one or more of the implants 500 of FIGS. 5 and/or 600 of FIG. 6. However, the struts 803 of the implant 800 at the flange 820 may be different from those of the implants 500 and 600. As illustrated in FIG. 8, the frame 802 may include multiple struts 803 (such as the struts 803a-803j) that project out and away from the frame 802 to facilitate creation of the flange 820. In doing so, the struts 803 may project away from a contact point with a remainder of the frame 802 in a spiral manner. Or stated another way, the series of struts 803 may create the visual effect of a spiral by the manner in which the struts 803 extend away from the remainder of the frame 802.

By providing the struts 803 in the spiral shape, the struts 803 may create the flange 820 of a similar or comparable size to that illustrated in FIGS. 5 and 6, but the flange 820 formed by the spiral-shaped struts 803 may provide for additional rigid structure within the flange 820 itself. For example, instead of projecting directly away from the frame to the edge of the flange in a directly radially outward direction from the frame (e.g., as illustrated in FIGS. 5 and 6), the struts 803 may sweep in both a radially outward and a circumferential direction about the generally circular shape 819 created by the edge of the frame 802 at the outer edge of the valve seat 18. Because the struts 803 extend away from the frame 802 in the spiral manner illustrated, the flange 820 may be able to provide a more consistent rigidity throughout the flange 820, and thus may provide a better seal as compared to the implants illustrated in FIGS. 5 and 6 against the anatomy of a patient. Additionally or alternatively, the flange 820 may be better suited to accommodate variations in anatomy as the flange may press in a more consistent manner around the outer edge of the flange 820 to create a seal against a variety of contours of anatomy.

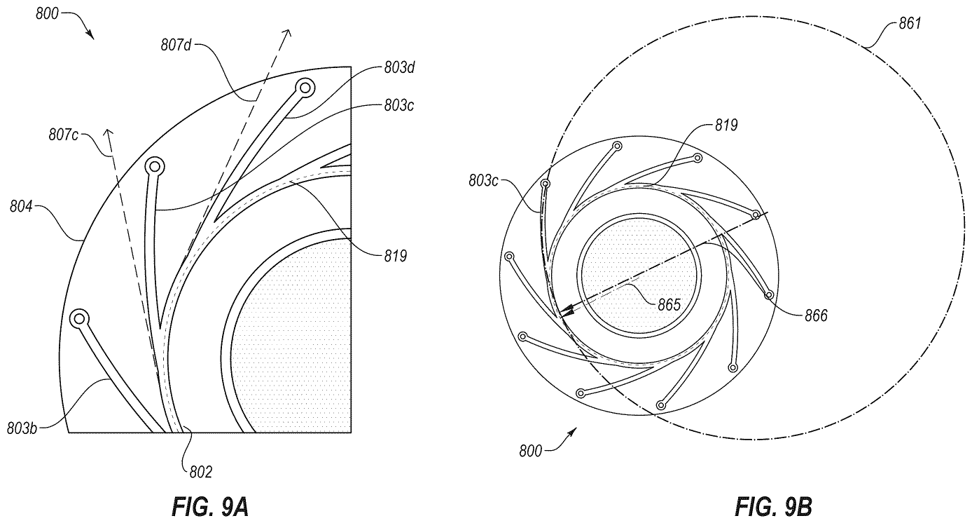

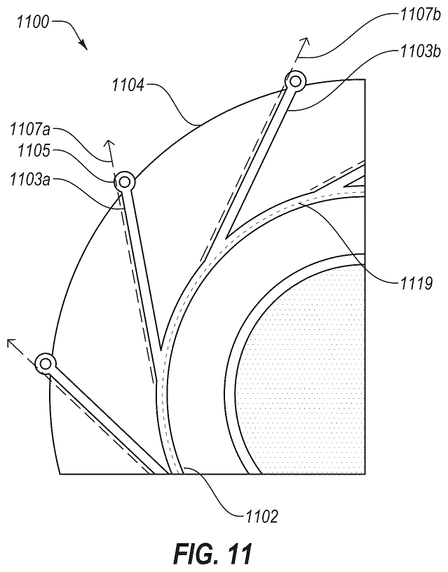

In some embodiments, the struts 803 may project away from the generally circular shape 819. For example, the struts 803 may project away in generally tangential directions away from the circular shape 819. In some embodiments, the tangential directions may include a lateral curvature (which may be described and explained in greater detail with reference to FIGS. 9A and 9B) or may include a generally straight profile (which may be described and explained in greater detail with reference to FIG. 11).

In some embodiments, the struts 803 may include nodes 805 at distal ends of the struts 803. For example, the nodes 805 may include a circular shape or bulge in material at the end of the struts 803. In some embodiments, the nodes 805 may include holes 806 via which the sealing element 804 may be sutured or otherwise fixedly attached to the struts 803. In some embodiments, the nodes 805 may reduce, control, or mitigate an amount of tissue penetration that the distal ends of the struts 803 cause. For example, when deploying the implant 800, the distal end of the struts 803 may bite into or otherwise penetrate the tissue of the patient when the implant 800 is being seated. By providing the nodes 805, that tissue penetration may be controlled.

In some embodiments, the nodes 805 may include a widening of the material of which the frame 802 is made. For example, the struts 803 may be a continuation of the frame 802 and the nodes 805 may be integrally formed with the struts 803. In some embodiments, the frame 802, including the struts 803 and the nodes 805 may be made of nitinol or other shape-memory alloy and cut from a single tube.

In some embodiments, the nodes 805 may include a coating, material, pad, foam, or other feature added to the distal end of the struts 803 to facilitate control of the amount of tissue penetration by the struts 803. While illustrated as being on each of the struts 803, it will be appreciated that a limited number of struts 803 may include the nodes 805. For example, every other strut 803 or every third strut 803 may include a node rather than each strut 803 including a node.

In some embodiments, the implant 800 may include one or more radiopaque markers to facilitate guidance, orientation, and otherwise facilitate a procedure of guiding the implant 800 through the body of the patient to the target location, deploying the implant 800, and/or otherwise positioning the implant 800 in the desired position. For example, the radiopaque markers may facilitate a view into an axial or radial orientation, state of deployment, or other information for the clinician when performing a procedure involving the implant 800. In some embodiments, the radiopaque markers may be located on the nodes 805. For example, the nodes 805 may include a radiopaque material being coined into the node 805, such as tantalum, bismuth, iodine, barium, or gold. In some embodiments, the holes 806 may be partly or fully filled with the radiopaque markers.

In some embodiments, each of the nodes 805 may include the radiopaque markers. Additionally or alternatively, fewer than all of the nodes 805 include the radiopaque markers. For example, every other node 805, every third node 805, every fourth node 805, etc. may include the radiopaque markers. As another example, every third node 805, every fourth node 805, etc. may not include the radiopaque markers and the remainder of the nodes 805 may include the radiopaque markers.

FIG. 9A illustrates a portion of the implant 800 of FIG. 8, in accordance with one or more embodiments of the present disclosure. FIG. 9A illustrates example tangent lines 807 (such as the tangent lines 807c and 807d) extending from the circular shape 819. For example, the tangent lines 807 may represent tangent lines of the circular shape 819 at the contact points at which the associated strut 803 extends from the frame 802.

As illustrated in FIG. 9A, the strut 803c extends from the frame 802 in a generally tangential direction that generally follows the tangent line 807c at a contact point where the strut 803c meets the remainder of the frame 802, and the strut 803d extends from the frame 802 in a generally tangential direction that generally follows the tangent line 807d. However, the struts 803 may include a lateral curvature that deviates from the tangent lines 807.

FIG. 9B illustrates the implant 800 of FIG. 8 with various measurements and shapes illustrated. As illustrated in FIG. 9B, a circle 861 defined by the radius of curvature of the strut 803c is illustrated relative to the circular shape 819. Additionally, the radius 865 of the circular shape 819 is illustrated relative to the radius 866 of the circle 861. In these and other embodiments, FIG. 9B illustrates that the radius of curvature of the struts 803 defining the lateral curvature of the struts 803 may be larger than the radius of curvature of the circular shape 819. In these and other embodiments, the spiral shape of the struts 803 may be formed by the struts 803 projecting off of the frame 802 in a direction generally following tangent lines 807 (such as illustrated in FIG. 9A) but with a radius of curvature as illustrated in FIG. 9B.

While a given radius of curvature and profile is illustrated in FIGS. 8, 9A, and 9B, it will be appreciated that the struts 803 may include a profile with a larger or smaller radius of curvature. Additionally or alternatively, while a given length of strut 803 is illustrated, it will be appreciated that the struts 803 may extend for any length. With a shorter length and a larger radius of curvature for the struts 803, the flange 820 may be less stiff while a smaller radius of curvature and longer length struts 803 may result in a stiffer flange 820. Additionally, while all of the struts 803 are illustrated of a uniform length, it will be appreciated that struts 803 of varying lengths are contemplated (e.g., four struts of a first length, four struts of a second length longer than the first length, and four struts of a third length longer than the second length).

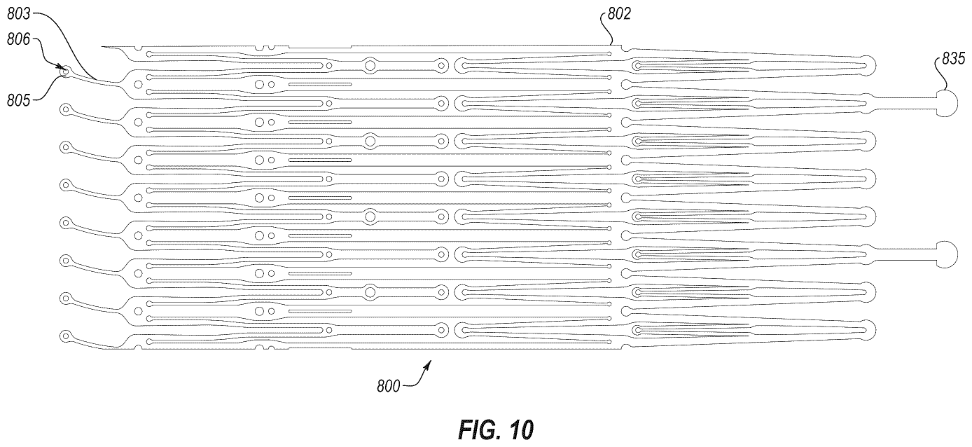

FIG. 10 illustrates an example of the implant 800 of FIG. 8, in accordance with one or more embodiments. For example, the implant 800 may depict an embodiment when cut or etched from a cylindrical tube. The view illustrated in FIG. 10 depicts the tube sliced in a longitudinal direction and unrolled such that an outer surface of the implant 800 that had a tubular shape is in a single plane for the convenience of visualization. It will be appreciated that in implementation, the tube may be cut or etched and remain in the tubular shape.

As illustrated in FIG. 10, the implant 800 may be cut or etched in a compressed state to facilitate the implant 800 traversing the body of the patient to the delivery location (e.g., the IVC, right atrium, etc.). As may be seen in FIG. 10, in a compressed state before being deployed, the struts 803 may be disposed on a distal end of the implant and may be generally parallel to each other when in the compressed/delivery state.

In some embodiments, the frame 802 of the implant 800 may include one or more feet 835 at a proximal end of the implant 800. The feet 835 may be used to detachably couple the implant 800 to a delivery device, such as a catheter or delivery capsule. Before delivery, the feet 835 may engage the delivery device, and after or during deployment, the feet 835 may be disengaged from the delivery device.

FIG. 11 illustrates another example of an implant 1100 configured to dock and/or support one or more prosthetic valves and/or valve components. The implant 1100 may be comparable or similar to the implant 800 of FIG. 8. For example, the implant 1100 may include a frame 1102, struts 1103, and a sealing element 1104, among other elements, that may be similar or comparable to the frame 802, struts 803, and sealing element 804 of the implant 800 of FIG. 8.

As illustrated in FIG. 11, the struts 1103 may extend away from the frame 1102 at tangential lines 1107 from a circular shape 1119 that may be comparable or similar to the circular shape 819 of the implant 800. The struts 1103 may extend away from the frame 1102 in a generally linear manner rather than a curved manner. For example, the struts 1103 may follow or run parallel to the tangent lines 1107 (such as the strut 1103a following the tangent line 1107a and the strut 1103b following the tangent line 1107b).

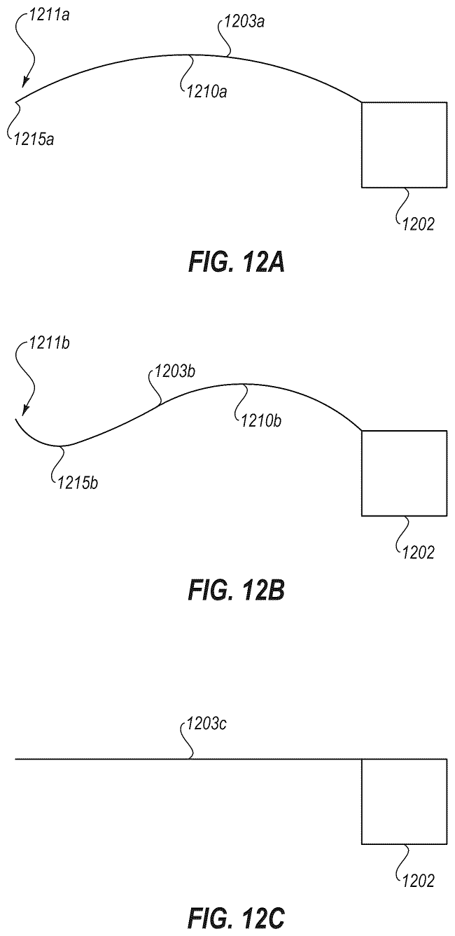

FIGS. 12A-12C illustrate examples of struts 1203, namely, the struts 1203a, 1203b, and 1203c, respectively. FIGS. 12A-12B illustrate the struts 1203 from a side view as they project away from a frame 1202. The struts 1203 may be consistent with and/or applicable to any of the other embodiments of the present disclosure. For example, the struts 803 of FIGS. 8, 9A, 9B, 10 and/or the struts 1103 of FIG. 11 may include a profile from a side view as disclosed in FIGS. 12A-12C.

As illustrated in FIG. 12A, the strut 1203a may include a curved profile when viewed from the side. The strut 1203a may initially rise up and away from the rest of the frame 1202 until it reaches an apex 1210a. After reaching the apex 1210a, the strut 1203a may extend downwards until a low point 1215a at the distal tip of the strut 1203a.

In some embodiments, the curved profile of the strut 1203a may provide for a more rigid structure within an associated flange. Additionally or alternatively, the curved profile of the strut 1203a may facilitate formation of a seal against the anatomy of a patient due to a spring effect from the curve of the strut 1203a that a clinician may pull against (e.g., the seating illustrated in FIG. 7E).

As illustrated in FIG. 12B, the strut 1203b may include a curved profile when viewed from the side. The strut 1203b may initially rise up and away from the rest of the frame 1202 until it reaches an apex 1210b. After reaching the apex 1210b, the strut 1203b may extend downwards until a low point 1215b. After reaching the low point 1215b, the strut 1203b may rise again until it reaches the distal tip 1211b of the strut 1203b. In some embodiments, the distal tip 1211b may be higher than the apex 1210b or lower than the apex 1210b.

By providing curved profile of the strut 1203b, an associated flange may enjoy similar benefits of rigidity and spring force articulated for the strut 1203a. Additionally or alternatively, the inverse curve towards the distal tip 1211b of the strut 1203b may reduce or minimize the amount of tissue penetration caused by the distal tip 1211 of the strut 1203.

As illustrated in FIG. 12C, in some embodiments, the profile of the strut 1203c may include a relatively flat profile when viewed from the side.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1

An implant comprising: an expandable frame configured to expand from a compressed state to an expanded state when deployed, the expandable frame including a longitudinal direction along a central axis through a central cavity formed by the expandable frame; and a plurality of struts at a first end of the expandable frame, the plurality of struts extending away from the expandable frame in a series of generally tangential directions away from a circular shape, the circular shape within the central cavity, normal to the longitudinal direction, and generally centered around the central axis.

### Example 2

Any of the foregoing examples, such as example 1, further comprising a skirt of material overlaying the plurality of struts and extending into the central cavity of the expandable frame.

### Example 3

Any of the foregoing examples, such as any of examples 1-2, further comprising a valve deployed within the central cavity.

### Example 4

Any of the foregoing examples, such as example 3, wherein the circular shape corresponds to a valve seat within which the valve is deployed.

### Example 5

Any of the foregoing examples, such as any of examples 1-4, wherein the generally tangential directions include curvature in a same direction as the circular shape and with a larger radius of curvature than that of the circular shape.

### Example 6

Any of the foregoing examples, such as any of examples 1-5, wherein the generally tangential directions include a lateral curvature in a same direction as the circular shape and with a larger radius of curvature than that of the circular shape.

### Example 7

Any of the foregoing examples, such as any of examples 1-6, wherein the plurality of struts form a flange at the first end of the expandable frame extending radially outward from the central axis beyond a remainder of the expandable frame.

### Example 8

Any of the foregoing examples, such as any of examples 1-7, wherein a given strut of the plurality of struts includes a curvature from a contact point at the expandable frame to an apex and from the apex to a distal end of the given strut, the curvature rising in the longitudinal direction as the given strut extends away from the expandable frame to the apex, and the curvature descending in the longitudinal direction from the apex to the distal end of the given strut.

### Example 9

Any of the foregoing examples, such as any of examples 1-8, wherein a given strut of the plurality of struts includes a curvature from a contact point at the expandable frame to an apex, from the apex to a low point, and from the low point to a distal end of the given strut, the curvature rising in the longitudinal direction as the given strut extends away from the expandable frame to the apex, the curvature descending in the longitudinal direction from the apex to the low point, and the curvature rising from the low point to the distal end of the given strut.

### Example 10

Any of the foregoing examples, such as any of examples 1-9, wherein a given strut of the plurality of struts includes a node at a distal end of the given strut.

### Example 11

Any of the foregoing examples, such as example 10, wherein the node includes an opening via which a skirt of material is coupled to the given strut using sutures.

### Example 12

Any of the foregoing examples, such as any of examples 10-11, wherein the node has a larger width than an arm of the given strut.

### Example 13

Any of the foregoing examples, such as any of examples 10-12, further comprising a skirt of material overlaying the plurality of struts and extending into the central cavity of the expandable frame, the skirt of material disposed on an outside of the node such that when deployed, the skirt of material is between the node and anatomy of a patient.

### Example 14

Any of the foregoing examples, such as any of examples 10-13, wherein the node has a pad on the node.

### Example 15

Any of the foregoing examples, such as examples 14, wherein the pad is made of a same material as a skirt of material overlaying the plurality of struts.

### Example 16

Any of the foregoing examples, such as any of examples 1-15, wherein the plurality of struts are integrally formed with the expandable frame.

### Example 17

Any of the foregoing examples, such as any of examples 1-16, wherein the expandable frame includes a plurality of vertical struts spanning between a proximal end and a distal end of the expandable frame.

### Example 18

Any of the foregoing examples, such as example 17, wherein the plurality of struts interface with the expandable frame at the distal end of the frame.

### Example 19

Any of the foregoing examples, such as any of examples 17-18, wherein the plurality of struts extend from where the vertical struts meet the distal end of the frame.

### Example 20

Any of the foregoing examples, such as any of examples 17-19, wherein the expandable frame includes a foot extending from the proximal end of the expandable frame.

### Example 21

Any of the foregoing examples, such as any of examples 1-20, wherein the plurality of struts are each a same length.

### Example 22

A method comprising: extending a delivery capsule through a patient in a first direction to a target location; at least partially deploying an implant at the target location, the implant including an expandable frame and a plurality of spiral struts extending away from the expandable frame in a spiral shape; and disengaging the implant from the delivery capsule.

### Example 23

Any of the foregoing examples, such as example 22, further comprising pulling the implant back in a second direction opposite of the first direction to seat the plurality of spiral struts against anatomy of the patient to create a seal between the implant and the anatomy.

### Example 24

Any of the foregoing examples, such as example 23, further comprising, after seating the plurality of spiral struts against the anatomy, completing any remaining deployment of the implant.

### Example 25

Any of the foregoing examples, such as example 24, wherein completing any remaining deployment of the implant comprises releasing a foot of the expandable frame from the delivery capsule and retracting the delivery capsule back through the patient.

### Example 26

Any of the foregoing examples, such as any of examples 23-25, wherein the seal is formed by a skirt of material affixed to the implant around the spiral struts and extending at least to a valve seat within the expandable frame.

### Example 27

Any of the foregoing examples, such as any of examples 23-26, further comprising installing a valve within the expandable frame at a valve seat within the expandable frame.

### Example 28

Any of the foregoing examples, such as example 27, wherein installing the valve comprises: extending a valve delivery capsule with the valve loaded thereon through the patient; deploying the valve within the implant; and fixing the valve to the implant in the valve seat.

### Example 29

Any of the foregoing examples, such as any of examples 10-15, wherein the node includes a radiopaque marker.

### Example 30

Any of the foregoing examples, such as any of examples 1-20, wherein each of the plurality of struts includes a node at a distal end of a respective strut.

### Example 31

Any of the foregoing examples, such as example 30, wherein each of the nodes includes a radiopaque marker.

### Example 32

Any of the foregoing examples, such as example 30, wherein fewer than each of the nodes includes a radiopaque marker.

### Example 33

Any of the foregoing examples, such as example 30, wherein every other of the nodes includes a radiopaque marker.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope and spirit of these claims.

1. An implant comprising:
an expandable frame configured to expand from a compressed state to an expanded state when deployed, the expandable frame including a longitudinal direction along a central axis through a central cavity formed by the expandable frame; and
a plurality of struts at a first end of the expandable frame, the plurality of struts extending away from the expandable frame in a series of generally tangential directions away from a circular shape, the circular shape within the central cavity, normal to the longitudinal direction, and generally centered around the central axis.

2. The implant of claim 1, further comprising a skirt of material overlaying the plurality of struts and extending into the central cavity of the expandable frame.

3. The implant of claim 2, further comprising a valve deployed within the central cavity.

4. The implant of claim 1, wherein a given strut of the plurality of struts includes a curvature from a contact point at the expandable frame to an apex and from the apex to a distal end of the given strut, the curvature rising in the longitudinal direction as the given strut extends away from the expandable frame to the apex, and the curvature descending in the longitudinal direction from the apex to the distal end of the given strut.

5. The implant of claim 1, wherein a given strut of the plurality of struts includes a curvature from a contact point at the expandable frame to an apex, from the apex to a low point, and from the low point to a distal end of the given strut, the curvature rising in the longitudinal direction as the given strut extends away from the expandable frame to the apex, the curvature descending in the longitudinal direction from the apex to the low point, and the curvature rising from the low point to the distal end of the given strut.

6. The implant of claim 1, wherein a given strut of the plurality of struts includes a node at a distal end of the given strut.

7. The implant of claim 6, wherein the node includes an opening via which a skirt of material is coupled to the given strut using sutures.

8. The implant of claim 6, wherein the node has a larger width than an arm of the given strut.

9. The implant of claim 6, further comprising a skirt of material overlaying the plurality of struts and extending into the central cavity of the expandable frame, the skirt of material disposed on an outside of the node such that when deployed, the skirt of material is between the node and anatomy of a patient.

10. The implant of claim 6, wherein the node has a pad on the node.

11. The implant of claim 6, wherein the node includes a radiopaque marker.

12. The implant of claim 1, wherein each of the plurality of struts includes a node at a distal end of a respective strut.

13. The implant of claim 12, wherein fewer than each of the nodes includes a radiopaque marker.

14. The implant of claim 1, wherein the plurality of struts are integrally formed with the expandable frame.

15. The implant of claim 1, wherein the expandable frame includes a plurality of vertical struts spanning between a proximal end and a distal end of the expandable frame.

16. The implant of claim 15, wherein the plurality of struts interface with the expandable frame at the distal end of the frame.

17. The implant of claim 16, wherein the plurality of struts extend from where the vertical struts meet the distal end of the frame.

18. The implant of claim 15, wherein the expandable frame includes a foot extending from the proximal end of the expandable frame.

19. A method comprising:
extending a delivery capsule through a patient in a first direction to a target location;
at least partially deploying an implant at the target location, the implant including an expandable frame and a plurality of spiral struts extending away from the expandable frame in a spiral shape; and
disengaging the implant from the delivery capsule.

20. The method of claim 19, further comprising pulling the implant back in a second direction opposite of the first direction to seat the plurality of spiral struts against anatomy of the patient to create a seal between the implant and the anatomy.
